(19) United States
(12) Patent Application Publication
(10) Pub. No.: US 2026/0345978 A1
(43) Pub. Date: Nov. 5, 2026

(54) MEDICAL DEVICE FOR LOCALIZED TREATMENT

(71) Applicant: Ignite Innovation Lab, LLC, Washington, DC (US)

(72) Inventors: Gabriel Fernando Santiago, Washington, DC (US); Maisie Margaret Mahoney, Boston, MA (US)

(73) Assignee: Ignite Innovation Lab, LLC, Washington, DC (US)

(21) Appl. No.: 19/194,674

(22) Filed: Apr. 30, 2025

(51) Int. Cl. A61M 37/00

(52) U.S. Cl. CPC A61M 37/00; A61M 2202/0007; A61M 2210/0687

(57) ABSTRACT

A device for localized treatment includes a sponge locatable at the treatment site, a catheter connected to the sponge, an optional reservoir connected to the catheter, a tube/port system connected to the catheter either via the reservoir or directly if the reservoir is omitted, and a syringe connected to the tube/port system and usable to deliver treatment substance to the sponge via the tube/port system, the reservoir, and the catheter. When the device with reservoir is used to treat gioblastoma, the sponge is placed below the patient's scalp and cranium with the catheter extending through the cranium to the reservoir, which is placed between the cranium and scalp with the tube/port system connected to the reservoir through the scalp.

### BACKGROUND

### Field of the Disclosure

The present invention relates generally to the field of medical devices, and more specifically, but not exclusively, to medical devices for localized treatment of solid tumor cavities such as occur during the surgical treatment of brain tumors like glioblastoma and sarcoma tumors.

### Description of the Related Art

This section introduces aspects that may help facilitate a better understanding of the disclosure. Accordingly, the statements of this section are to be read in this light and are not to be understood as admissions about what is prior art or what is not prior art.

Following surgical removal of brain neoplasms, treatment often involves a combination of therapeutic modalities such as systemic chemotherapy and radiation therapy to address potential residual tumor cells and prevent recurrence. Glioblastoma, a brain neoplasm characterized by its intricate and aggressive nature, presents formidable challenges in treatment owing to its irregular patterns and its occurrence within the intricate framework of the brain's delicate tissues. Standard-of-care treatment using systemic chemotherapy can lead to a range of significant side effects, including nausea, fatigue, hair loss, and compromised immune function, which can impact the patient's overall quality of life. Local medication delivery strategies, exemplified by the deployment of Gliadel® Wafers, offer a localized therapeutic approach by gradually dispensing medications directly at the tumor site. Gliadel® Wafers are manufactured by Eisai Inc. for Azurity Pharmaceuticals, Inc., Baltimore, MD, USA. Nevertheless, the disc shape and temporary nature of these wafers, which gradually dissolve and discontinue drug delivery, underscores the urgency for a more-enduring and more-efficacious method for localized treatment.

### SUMMARY

This disclosure introduces an innovative device that addresses this unmet need by establishing a port beneath the scalp, connected to a conduit leading to a sponge embedded within a tumor cavity post excision. While initially conceived for the context of glioblastoma treatment, the versatility of this device extends far beyond a singular tumor type. The device's adaptability renders it suitable for facilitating localized medication delivery across a spectrum of brain neoplasms, or any other solid organ tumor in which a cavity (or potential cavity) results following surgical or radiation therapy treatment, or any cavity that would benefit from highly localized treatment at the cavity site, regardless of their distinct characteristics or anatomical locations. The key feature of this device is the sponge at the cavity site, so medication delivery to the sponge may vary by treatment type to not include a reservoir, but to still include the general process as described in this patent.

By offering an enduring avenue for precise drug administration, this inventive solution has the potential to revolutionize the treatment landscape for diverse brain tumors. As opposed to the transitory intervention of conventional wafers, or convection enhanced medication delivery, this device signifies a paradigm shift towards consistent and comprehensive localized therapy. The ensuing sections expand upon the technical aspects and operational mechanisms of this device, underscoring its immense potential to transform the landscape of brain neoplasm treatment.

In addition to overcoming the limitations of fixed-dose delivery systems like Gliadel® Wafers, a device of this disclosure offers one or more of the following key clinical and therapeutic advantages. First, it enables repeat dosing via a subcutaneous port, allowing for ongoing, titratable drug delivery that can be adjusted over time to match patient-specific treatment responses. This approach supports real-time therapy customization based on imaging results, biomarker levels, or clinical symptoms—an important step toward truly personalized medicine in neuro-oncology. Moreover, because the port is implanted beneath the scalp and accessed percutaneously, the system allows for minimally invasive re-dosing without the need for additional craniotomies or complex surgical interventions. The hybrid design, which includes an optional Ommaya reservoir, provides flexibility for either scheduled bolus infusions or continuous slow-drip administration, depending on the therapeutic protocol. Lastly, the delivery system is compatible not only with chemotherapeutic agents, but also with immunotherapies, biologics, and radiopharmaceuticals, offering a promising platform for delivering treatments that are otherwise too toxic, unstable, or inefficient when administered systemically.

The device shown in FIG. 1A provides a novel solution to this problem. The device 100 comprises a syringe 1, a tube/port system 2, an (optional) Ommaya reservoir 5, a catheter 8, and a biocompatible sponge 6. As shown in FIG. 2, the sponge 6 is positioned at the tumor site and is designed to absorb and release specific drugs delivered through the tube/port system 2 connected to the syringe 1.

The device 100 is designed to deliver chemotherapy or other anti-neoplastic drugs such as (without limitation) temozolomide, carmustine (BCNU), and/or lomustine (CCNU) for localized treatment. The materials used for the syringe 1, tube/port system 2, Ommaya reservoir 5, and sponge 6 are chosen for their biocompatibility and functionality. The optional Ommaya reservoir 5, made of silicone, can be used for prolonged drug delivery.

The surgical procedure for deploying the device 100 involves several steps including making an incision, creating burr holes in the skull 4 (FIG. 2), performing a craniotomy, opening the dura mater 7 (FIG. 2), and then placing the sponge 6 in the tumor resection cavity. After the sponge placement, the surgery concludes with dural closure, bone flap replacement, scalp closure, dressing, and patient recovery.

### BRIEF DESCRIPTION OF THE DRAWINGS

Embodiments of the disclosure will become more fully apparent from the following detailed description, the appended claims, and the accompanying drawings in which like reference numerals identify similar or identical elements.

FIG. 1A is a perspective view of the device according to one embodiment of the disclosure;

FIG. 1B is a perspective view of the unassembled device of FIG. 1A;

FIG. 1C is a perspective view of the tube/port system of FIG. 1A, according to certain embodiments;

FIG. 2 is a perspective view of the device of FIG. 1A deployed to treat a brain tumor;

FIG. 3A is a perspective view of the device of FIG. 1A being deployed during surgery; and

FIG. 3B is a perspective view of the device of FIG. 1A as deployed after the surgery is complete.

### DETAILED DESCRIPTION

Detailed illustrative embodiments of the present disclosure are disclosed herein. However, specific structural and functional details disclosed herein are merely representative for purposes of describing example embodiments of the present disclosure. The present disclosure may be embodied in many alternate forms and should not be construed as limited to only the embodiments set forth herein. Further, the terminology used herein is for the purpose of describing particular embodiments only and is not intended to be limiting of example embodiments of the disclosure.

As used herein, the singular forms “a,” “an,” and “the,” are intended to include the plural forms as well, unless the context clearly indicates otherwise. It further will be understood that the terms “comprises,” “comprising,” “contains,” “containing,” “includes,” and/or “including,” specify the presence of stated features, steps, or components, but do not preclude the presence or addition of one or more other features, steps, or components. It also should be noted that in some alternative implementations, the functions/acts noted may occur out of the order noted in the figures. For example, two figures shown in succession may in fact be executed substantially concurrently or may sometimes be executed in the reverse order, depending upon the functions/acts involved.

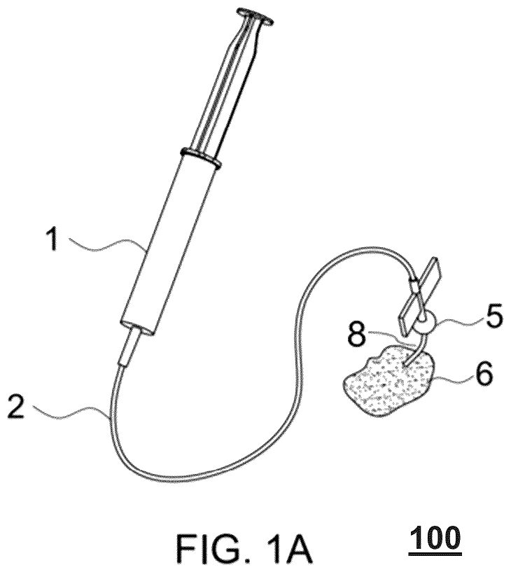

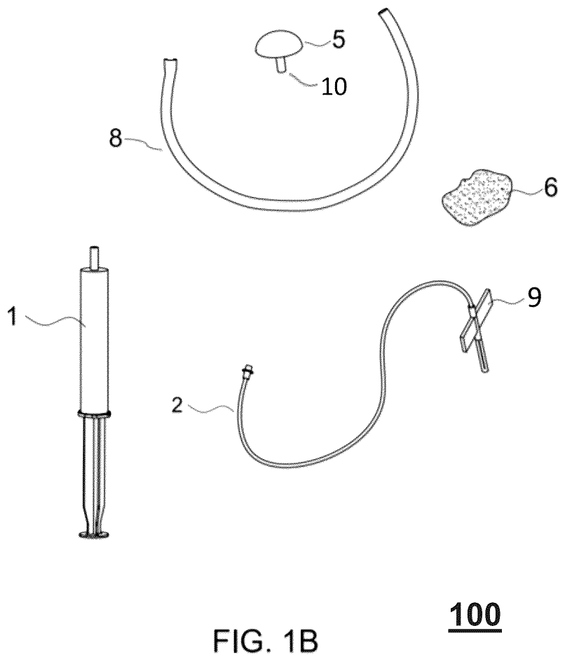

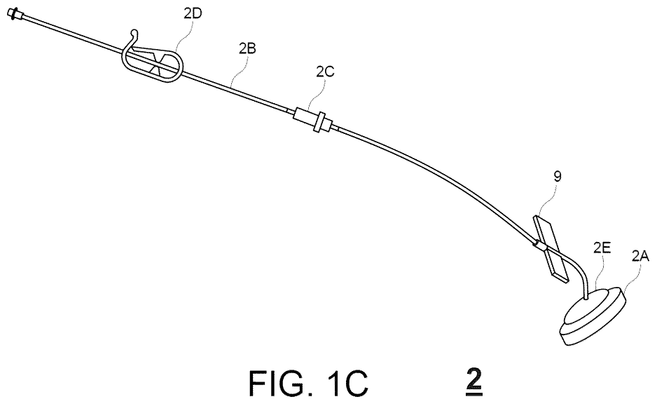

The device 100 of FIGS. 1A-1B is composed of three main parts: the sponge 6, the tube/port system 2, and the syringe 1. The sponge 6, potentially made of materials such as collagen, chitosan, or specific polymers, is designed to absorb and gradually release chemotherapy or other anti-neoplastic drugs at the tumor site. The tube/port system 2, possibly constructed from medical-grade silicone or polyurethane, is connected to the external syringe 1 and optionally to an Ommaya reservoir 5 for easy, repeat drug administration. The syringe 1 itself is typically made from plastic and rubber, compatible with the drug being administered.

The device 100 is designed to deliver chemotherapy or other anti-neoplastic drugs for the localized treatment of glioblastoma.

Syringe 1 delivers the chemotherapy or other anti-neoplastic drug into the sponge 6 through the tube/port system 2. The syringe 1 is designed to be compatible with various chemotherapy or other anti-neoplastic agents and is typically made from medical-grade plastics. The material choice for the syringe 1 can vary, but a biocompatible plastic, such as polypropylene, is generally preferred due to its durability, chemical resistance, and cost-effectiveness. The syringe 1 may also have gradation marks (not shown in the figures) to enable precise measurement of the drug volume. In alternative implementations, the syringe 1 may be replaced with a suitable device, such as an electromechanical pump, osmotic delivery system, or gravity-based infusion mechanism incorporating flow control features such as clamps or restrictors.

FIG. 1C is a perspective view of the tube/port system 2, according to certain embodiments. The tube/port system 2 connects the catheter 8 and the sponge 6 to the external syringe 1, facilitating the drug's delivery. The tube/port system 2 allows for the initial delivery of medication to the sponge 6 and potentially additional postoperative drug administrations. The tube/port system 2 comprises a subcutaneous port 2A accessible through the skin, flexible medical-grade tubing 2B, and an optional one-way valve 2C, a flow regulator 2D, and a self-sealing septum or injection dome 2E for reservoir access.

The tube/port system 2 should be flexible and robust enough to withstand repeated drug delivery procedures and should include (i) a one-way valve (not shown in figures) to prevent backflow, (ii) kink-resistant features to maintain a consistent drug delivery pathway, and (iii) a bio-compatible needle 9 to penetrate the skin to deliver drugs to the Ommaya reservoir 5. Kink-resistant features could include, but are not limited to, webbed tubing which has a mesh-like structure that prevents helical kinking, adding internal stiffeners or helical reinforcement, or incorporating a harder polymer into the tubing to act as a structural brace to prevent kinking. The needle 9 should be biocompatible material and should be approved for delivery of drugs to the material/mechanism chosen to receive drugs to then be delivered through the catheter 8 to the sponge 5. The tube/port system 2 may have translucency that allows for visual inspection of drug flow. Made from medical-grade materials, the tube/port system 2 ensures biocompatibility and durability. A suitable material would be medical-grade silicone, known for its flexibility, biocompatibility, and durability.

In some embodiments, the tube/port system 2 includes integrated flow-control elements such as a one-way valve or pressure-sensitive regulator to prevent reverse flow and accidental overdose. The system may be further equipped with antimicrobial coatings or self-sealing septa to minimize infection risk during repeated access.

The catheter 8 connects the Ommaya reservoir 5 to the sponge 6 via a suitable mechanical interface such as a barbed fitting, Luer-lock, or adhesive seal. At the sponge end, the catheter 8 may terminate in a needle or be embedded in a recessed groove or central bore designed to ensure stable, sealed delivery of medication. The exit port 10 of the reservoir 5 fits into one end of the catheter 8. The other end of the catheter 8 secures into the sponge 6. The sponge 6 is designed and placed so that there is a mechanism for the catheter 8 to securely attach and deliver the medication to the sponge. This could be, but is not limited to, a needle (not shown) at the end of the catheter 6 or a longitudinal central channel (not shown) in the sponge 6 accommodating the catheter 8.

The sponge 6 is the primary therapeutic component. Made from biocompatible materials, the sponge 6 is designed to absorb and gradually release chemotherapy or other anti-neoplastic drugs directly at the tumor site. The sponge's composition ensures it is gentle on surrounding brain or other tumor cavity tissue while effectively delivering medication. Features of the sponge that make it suitable to achieve these results include, but are not limited to:
Porous structure: the sponge 6 has a high surface area and porosity, which can be tailored to control the release rate of medications by adjusting the pore size and distribution.
Absorbent properties: the sponge 6 can absorb and retain fluids, which can help maintain the medication's concentration at the target site.
Biocompatible material: the sponge 6 is composed of a material that is selected for its softness and flexibility, ensuring that it conforms to the irregularities of the tumor cavity without causing undue pressure on the surrounding brain tissue. This material also reduces the likelihood of adverse reactions to the surrounding tissue due to its biocompatibility.

The sponge 6 sits in the tumor site, serving as the delivery point for the drug. The sponge 6 should be made of a material that can absorb and gradually release the drug over time. Bioresorbable polymers, such as polyglycolic acid (PGA), polylactic acid (PLA), or their copolymer polylactic-co-glycolic acid (PLGA), are excellent candidates. These materials are not only biocompatible and capable of drug delivery, but they also gradually degrade and get absorbed by the body over time, reducing the need for surgical removal of the sponge 6. The surgeon will, however, still need to complete the process of removing the tube/port system 2 from the brain in a process that is typically less invasive than the initial insertion. This procedure is less invasive than the initial procedure, as it does not require a new pathway to be created in the brain, although the specific procedure will depend on the surgeon and the patient's unique circumstances.

The leading theory is to have the sponge 6 fabricated from bioresorbable polymers like PLGA, which is known for its controlled degradation and drug-release properties. The sponge's porous structure is engineered to optimize drug absorption and release, ensuring a consistent therapeutic effect at the tumor site. The sponge's rate of biodegradation may be calibrated to match the treatment duration, gradually resorbing to minimize the need for surgical removal.

In some embodiments, the sponge 6 may be provided in a compressed or dehydrated state and designed to expand upon placement in the tumor cavity, thereby ensuring gentle and complete contact with the surrounding resection margins without exerting harmful pressure on brain tissue. The sponge may also be available in various pre-molded shapes for common surgical configurations or supplied in a format that allows for intraoperative trimming or shaping, enhancing its adaptability to a range of anatomical presentations. To support varying therapeutic needs, the size and volume of the sponge 6 can be adjusted, allowing for absorption of different quantities of medication, and it is designed to be refillable and reusable throughout the course of treatment. Advanced implementations may include multi-layered zones to facilitate staged or time-controlled drug release, microchannel networks to direct diffusion toward areas of concern, or surface coatings incorporating nanoparticles to improve drug penetration or target specificity. These enhancements offer clinicians increased control over therapeutic delivery, improving both precision and efficacy while reducing off-target effects.

The optional Ommaya reservoir 5, a dome-shaped device, can be used to deliver drugs to the sponge 6 over time. The reservoir 5, usually made of silicone, is surgically implanted beneath the scalp and connected to the sponge 6 via a catheter 8. The drugs are injected into the reservoir 5 through the scalp and are then delivered to the sponge 6. The reservoir is made of a self-sealing silicone, or a similar biocompatible material.

The exact choice of materials for the device of FIGS. 1A-1B may vary based on specific medical considerations, patient tolerance, desired rate of drug release, tumor cavity shape, and degradation time of the sponge 6.

### Procedure for Use

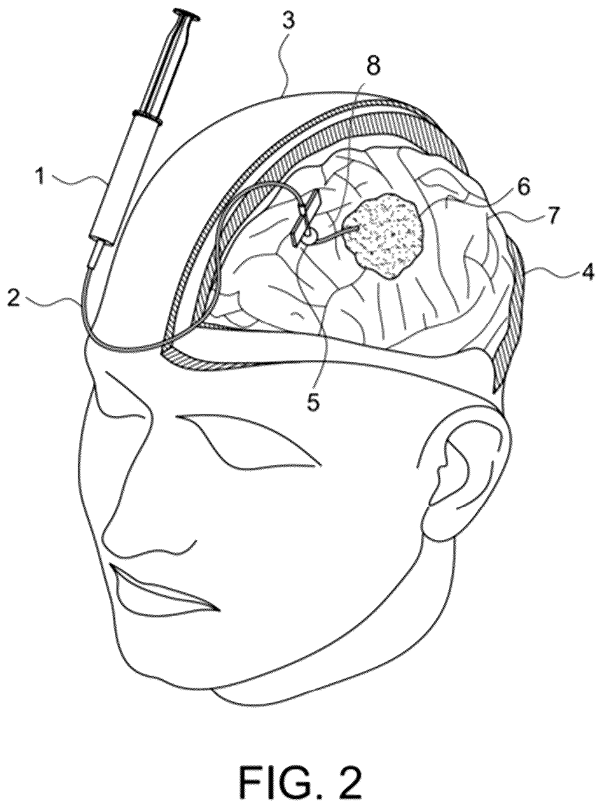

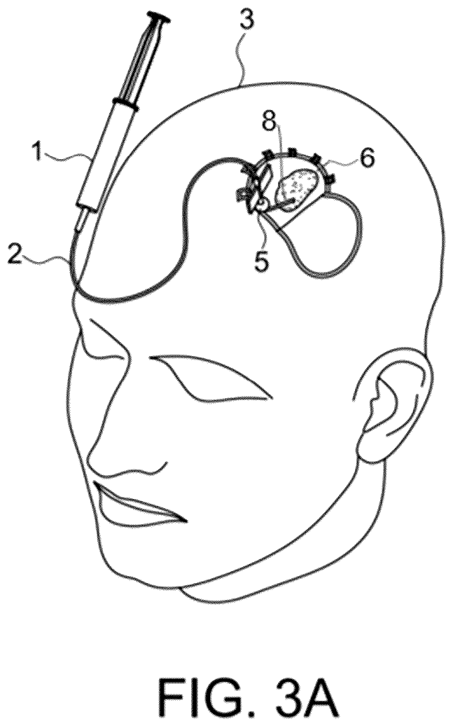

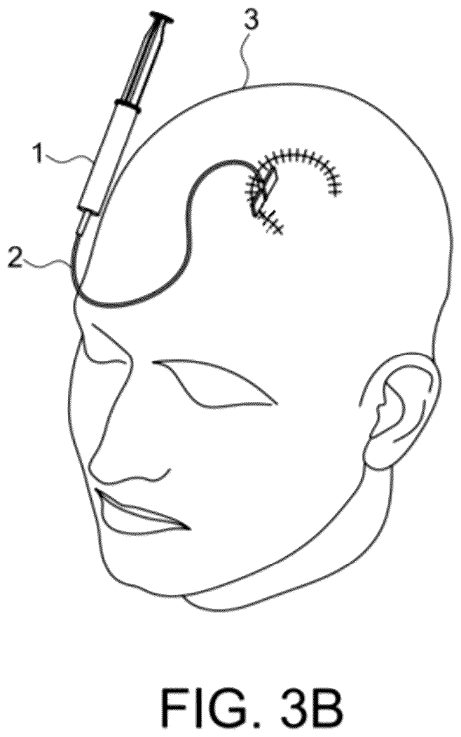

As represented in FIGS. 2 and 3A-3B, the device 100 may be deployed during surgical removal of a tumor. Following the removal of the tumor, the sponge 6 is inserted into the post-surgical site, and the tube/port system 2 is connected to the syringe 1.

In the surgical procedure involving the device 100, initially, a curvilinear incision is made on the patient's head, and the scalp flap is carefully elevated to reveal the skull. Following this, burr holes are drilled into the skull, and a craniotomy is performed to access the brain. Once the brain is exposed, the dura mater is incised, providing access to the brain tissue where the surgical intervention, such as tumor resection, takes place.

The key step involves the placement and securing of the device 100. In some implementations, after the surgical intervention, the unassembled device 100 shown in FIG. 1B is removed from its sterile packaging and assembled. The device's functionality may be tested by injecting saline into its port. After confirming its operational readiness, the sponge 6 is then delicately placed into the tumor resection cavity. The catheter 8 of the device 100 is meticulously brought out through one of the previously drilled burr holes. The mechanism of drug release involves diffusion through the sponge's porous matrix, allowing for a sustained release of the chemotherapy or other anti-neoplastic drugs at the targeted site. As understood by those skilled in the art, the port 2A may have flanges (not shown) that enable the device 100 to be secured in the resection cavity with titanium screws, chosen for their biocompatibility, strength, and MRI compatibility, or another accepted method of securing the tubing/port system 2. This ensures the device 100 remains fixed in place during the entire drug delivery period and minimizes any potential movement or displacement. Following the deployment of the device 100, the procedure concludes with meticulous steps to ensure patient safety and promote healing. The dura mater is sutured closed in a watertight manner, and the bone flap, previously removed, is repositioned and secured with titanium miniplates and screws.

The scalp flap is then carefully reapproximated and closed in layers to minimize scarring and promote healing. A sterile dressing and a closed suction drain are applied to the incision site to manage postoperative healing. The patient is then carefully monitored as they recover from anesthesia and are subsequently moved to a post-anesthesia care unit for further observation and care. This detailed and careful process aims to ensure the effective deployment of the device 100 for localized drug delivery while minimizing complications and promoting patient recovery.

As indicated in FIGS. 3A and 3B, after the surgery is completed, the sponge 6 is located inside the patient's skull 3 and the Ommaya reservoir 5 is located outside the skull 3 but under the scalp, with the interconnecting catheter 8 extending through an opening in the skull and attaching to the Ommaya reservoir (or similar port for implementations that do not involve an Ommaya reservoir).

For the delivery of medication over time, the syringe 1 may be attached and re-attached and does not need to remain permanently fastened to the device. In configurations with the reservoir 5, the syringe 1 and tube/port system 2 accesses the reservoir 5 through a subcutaneous injection dome of the reservoir 5. In embodiments where the reservoir 5 is omitted, the tube/port system 2 connects directly to the catheter 8. This connection may be achieved via a suitable mechanism such as a medical-grade coupler, adhesive, or friction fit, ensuring fluid-tight delivery to the sponge 6. The frequency of using the syringe 1 to deliver medication depends on the patient's treatment plan and the specific medication being administered.

In some implementations, the sponge 6 may be designed for retrieval following completion of therapy. In such cases, the sponge 6 is fabricated from a non-degradable, biocompatible material and positioned in a way that allows for access via the original surgical pathway or through an extended catheter 8. The sponge 6 may include an integrated retrieval feature, such as a tether, retrieval tab, or embedded magnet, which can be accessed endoscopically, percutaneously, or during a follow-up craniotomy. This retrievability option enables clinicians to remove the device if necessary due to infection, treatment changes, or completion of therapy, and provides added flexibility in long-term treatment planning.

Although the technology has been described in the context of the embodiment shown in the figures in which medication is delivered to the sponge 6 using an assembly that includes the syringe 1, the tube/port system 2, the Ommaya reservoir 5, and the catheter 8, other suitable techniques may be employed to deliver medication to an embedded sponge including, for example (without limitation), using a pump or gravity-based device instead of a syringe and with or without a subcutaneous reservoir such as the Ommaya reservoir 5.

Although primarily designed for intracranial tumor cavities, the device may be adapted for use in other solid-organ tumor resections where a cavity remains, including but not limited to hepatic, pancreatic, renal, muscle, and pulmonary tissues. The device may also be adapted for use in other applications requiring administration of medication such as in wound healing or regenerative medicine.

In certain embodiments, the present disclosure is a device for localized medical treatment, the device comprising a sponge, a catheter connectable to the sponge, and a tube/port system connectable between the catheter and a syringe. The sponge is configured to be placed at a patient's treatment site, and the tube/port system is configured to deliver a treatment substance from the syringe to the treatment site via the catheter and the sponge.

In at least some of the above embodiments, the treatment site is at the patient's brain; the sponge is configured to be placed at the treatment site inside the patient's cranium; and the catheter is configured to extend from the sponge through the patient's cranium.

In at least some of the above embodiments, the device further comprises a reservoir connectable between the catheter and the tube/port system, wherein the reservoir is configured to store a supply of the treatment substance.

In at least some of the above embodiments, the treatment site is at the patient's brain; the sponge is configured to be placed at the treatment site inside the patient's cranium; the reservoir is configured to be placed outside the patient's cranium and inside the patient's scalp; and the catheter is configured to extend from the sponge through the patient's cranium to the reservoir; and the tube/port system is configured to extend through the patient's scalp to the reservoir.

In at least some of the above embodiments, the device of claim 1, further comprises the syringe.

In certain embodiments, the present disclosure is a method for localized medical treatment using the assembled device of claim 1, wherein the method comprises: opening the patient's scalp and cranium to expose the treatment site; placing the sponge at the treatment site; closing the patient's cranium and scalp with the catheter extending through the cranium; connecting the tube/port system to the catheter; and using the syringe to deliver the treatment substance to the sponge via the tube/port system and the catheter.

In certain embodiments, the present disclosure is a method of localized treatment using the assembled device of claim 3, wherein the method comprises opening the patient's scalp and cranium to expose the treatment site; placing the sponge at the treatment site with the sponge connected to the catheter; closing the patient's cranium with the catheter extending through the cranium and connected to the reservoir; placing the reservoir outside the patient's cranium with the catheter extending from the sponge through the patient's cranium to the reservoir; closing the patient's scalp with the reservoir located between the patient's cranium and the patient's scalp; connecting the tube/port system to the reservoir through the patient's scalp; and using the syringe to deliver the treatment substance to the reservoir via the tube/port system, such that the reservoir delivers the treatment substance to the sponge via the catheter.

In at least some of the above embodiments, the treatment substance is an anti-neoplastic drug.

In at least some of the above embodiments, the anti-neoplastic drug is for treatment of gioblastoma.

Unless explicitly stated otherwise, each numerical value and range should be interpreted as being approximate as if the word “about” or “approximately” preceded the value or range.

Reference herein to “one embodiment” or “an embodiment” means that a particular feature, structure, or characteristic described in connection with the embodiment can be included in at least one embodiment of the disclosure. The appearances of the phrase “in one embodiment” in various places in the specification are not necessarily all referring to the same embodiment, nor are separate or alternative embodiments necessarily mutually exclusive of other embodiments. The same applies to the term “implementation.”

Unless otherwise specified herein, the use of the ordinal adjectives “first,” “second,” “third,” etc., to refer to an object of a plurality of like objects merely indicates that different instances of such like objects are being referred to, and is not intended to imply that the like objects so referred-to have to be in a corresponding order or sequence, either temporally, spatially, in ranking, or in any other manner.

Also for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” etc., imply the absence of such additional elements. The same type of distinction applies to the use of terms “attached” and “directly attached,” as applied to a description of a physical structure. For example, a relatively thin layer of adhesive or other suitable binder can be used to implement such “direct attachment” of the two corresponding components in such physical structure.

The described embodiments are to be considered in all respects as only illustrative and not restrictive. In particular, the scope of the disclosure is indicated by the appended claims rather than by the description and figures herein. All changes that come within the meaning and range of equivalency of the claims are to be embraced within their scope.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps, where the additional, unrecited elements or steps do not have the one or more specified characteristics.

As used herein, “at least one of the following: <a list of two or more elements>” and “at least one of <a list of two or more elements>” and similar wording, where the list of two or more elements are joined by “and” or “or”, mean at least any one of the elements, or at least any two or more of the elements, or at least all the elements. For example, the phrases “at least one of A and B” and “at least one of A or B” are both to be interpreted to have the same meaning, encompassing the following three possibilities: 1—only A; 2—only B; 3—both A and B.

While preferred embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the technology of the disclosure. It is intended that the following claims define the scope of the invention and that methods and structures within the scope of these claims and their equivalents be covered thereby.

What is claimed is:

1. A device for localized medical treatment, the device comprising:
a sponge;
a catheter connectable to the sponge; and
a tube/port system connectable between the catheter and a syringe, wherein:
the sponge is configured to be placed at a patient's treatment site; and
the tube/port system is configured to deliver a treatment substance from the syringe to the treatment site via the catheter and the sponge.

2. The device of claim 1, wherein:
the treatment site is at the patient's brain;
the sponge is configured to be placed at the treatment site inside the patient's cranium; and
the catheter is configured to extend from the sponge through the patient's cranium.

3. The device of claim 1, further comprising a reservoir connectable between the catheter and the tube/port system, wherein the reservoir is configured to store a supply of the treatment substance.

4. The device of claim 3, wherein:
the treatment site is at the patient's brain;
the sponge is configured to be placed at the treatment site inside the patient's cranium;
the reservoir is configured to be placed outside the patient's cranium and inside the patient's scalp;
the catheter is configured to extend from the sponge through the patient's cranium to the reservoir; and
the tube/port system is configured to extend through the patient's scalp to the reservoir.

5. The device of claim 1, further comprising the syringe.

6. The device of claim 1, wherein the treatment substance is an anti-neoplastic drug.

7. The device of claim 6, wherein the anti-neoplastic drug is for treatment of gioblastoma.

8. A method for localized medical treatment using the assembled device of claim 1, wherein the method comprises:
opening the patient's scalp and cranium to expose the treatment site;
placing the sponge at the treatment site;
closing the patient's cranium and scalp with the catheter extending through the cranium;
connecting the tube/port system to the catheter; and
using the syringe to deliver the treatment substance to the sponge via the tube/port system and the catheter.

9. The method of claim 8, wherein the treatment substance is an anti-neoplastic drug.

10. The method of claim 9, wherein the anti-neoplastic drug is for treatment of gioblastoma.

11. A method of localized treatment using the assembled device of claim 3, wherein the method comprises:
opening the patient's scalp and cranium to expose the treatment site;
placing the sponge at the treatment site with the sponge connected to the catheter;
closing the patient's cranium with the catheter extending through the cranium and connected to the reservoir;
placing the reservoir outside the patient's cranium with the catheter extending from the sponge through the patient's cranium to the reservoir;
closing the patient's scalp with the reservoir located between the patient's cranium and the patient's scalp;
connecting the tube/port system to the reservoir through the patient's scalp; and
using the syringe to deliver the treatment substance to the reservoir via the tube/port system, such that the reservoir delivers the treatment substance to the sponge via the catheter.

12. The method of claim 11, wherein the treatment substance is an anti-neoplastic drug.

13. The method of claim 12, wherein the anti-neoplastic drug is for treatment of gioblastoma.
